(19) United States
(12) Patent Application Publication
(10) Pub. No.: US 2015/0088251 A1
(43) Pub. Date: Mar. 26, 2015

(54) CARDIAC VALVE PROSTHESIS

(71) Applicant: San Diego State University Research Foundation, San Diego, CA (US)

(72) Inventor: Karen May-Newman, San Diego, CA (US)

(73) Assignee: San Diego State University Research Foundation, San Diego, CA (US)

(21) Appl. No.: 14/490,374

(22) Filed: Sep. 18, 2014

Related U.S. Application Data
(60) Provisional application No. 61/883,091, filed on Sep. 26, 2013.

(51) Int. Cl. A61F 2/24

(52) U.S. Cl. CPC A61F 2/2412
USPC 623/2.12

(57) ABSTRACT

Provided herein is a prosthetic device in patients with ventricular assist device (VAD) support and a closed off aortic valve orifice, which reestablishes the formation of normal fluid flow patterns in the aortic root. The prosthesis includes a circular body having a first surface and a second surface and a projection disposed on the first surface of the body, wherein the projection includes a plurality of walls extending away from the first surface of the body, each wall being positioned such that a first side edge contacts a center of the body, thereby forming a plurality of intercepted arcs, each located between adjacent walls; and a plurality of concave parabolic surfaces, each disposed within an intercepted arc. Also provided are methods of using the prosthetic device to reduce thrombosis formation in patients with VAD support.

### CROSS REFERENCE TO RELATED APPLICATION(S)

This application claims the benefit of priority under 35 U.S.C. §119(e) of U.S. Ser. No. 61/883,091, filed Sep. 26, 2014, the entire content of which is incorporated herein by reference.

### BACKGROUND OF THE INVENTION

1. Field of the Invention

The invention relates generally to an implantable cardiac prosthesis, and more specifically to a cardiac valve prosthesis used in aortic valve closure surgery to improve the flow pattern in the aortic root.

2. Background Information

Heart failure is a debilitating condition that affects approximately 5.8 million Americans. Heart failure is characterized by poor cardiac function and the heart's inability to meet the body's demands. Heart transplants are in limited supply and come with long waiting-lists, often months to years to find donor matches.

Continuous flow left ventricular assist devices (LVADs) are implantable, mechanical pumps implanted to treat severe heart failure. LVADs can act as bridge-to-transplant until a suitable donor is found, or act as a permanent implant. Although LVADs have been shown to significantly improve mortality and quality of life, the alteration of the blood flow pathway creates high-risk complications, such as thromboembolism. Furthermore, persistent high transvalvular pressure across the aortic valve reduces its opening frequency, which tends to worsen any native aortic valve insufficiency. However, prosthetic aortic valves are contraindicated in LVAD patients, leaving approximately 20% of patients with conditions warranting permanent closure of the aortic valve. Surgical closure of the valve has demonstrated no adverse effects on mortality, but the closed pathway in the aortic root provides a greater risk for blood stasis, which may initiate thrombosis. The current practice for valve closure surgery is to use pericardial strips or patches, which provide a secure closure, but are not systematically designed to produce a consistent, long-term result.

The importance of the flow pattern in the aortic root has been long-appreciated. The aortic valve leaflets, the aortic root and sinus walls function together to direct flow in vortices that washout the aortic sinuses and feed the coronary arteries. Thus, there is a need for a device that reestablishes the normal flow patterns in the aortic root of patients that have a surgically closed aortic valve.

### SUMMARY OF THE INVENTION

The present invention is based on the design and utilization of a prosthetic device in patients with ventricular assist device (VAD) support and a closed off aortic valve orifice, which reestablishes the formation of normal fluid flow patterns in the aortic root. The formation of fluid vortices in the aortic root is considered important in the prevention of thrombus formation; thus, reestablishing advantageous flow patterns such as sinus vortices is the goal of the prosthetic design.

Accordingly, in one aspect there is provided a cardiac prosthesis including a circular body having a first surface and a second surface and a projection disposed on the first surface of the body, wherein the projection includes a plurality of walls extending away from the first surface of the body, each wall being positioned such that a first side edge contacts a center of the body, thereby forming a plurality of intercepted arcs, each located between adjacent walls; and a plurality of concave parabolic surfaces, each disposed within an intercepted arc. In some aspects, the projection includes three walls. In some embodiments, the second side edge of each wall inclines toward the center of the body. In certain aspects, the projection is substantially conical. In various embodiments, the top edge of each wall inclines from the center of the body toward the circumferential edge of the body. In some aspects, the top edge of each wall is rounded; in other aspects, the top edge of each wall is flat.

In various embodiments, the prosthesis includes a flange disposed along a circumferential edge of the body and extending away from the first surface of the body. In further embodiments, the prosthesis may include a gasket disposed on the second surface of the body and configured to sealingly attach the body to a cardiac valve. In various embodiments, the cardiac valve is surgically closed.

In some embodiments, the surface of the projection is smooth. In alternate embodiments, the surface of the projection is rough.

In various embodiments, the body and projection are formed from a synthetic inert material. In other embodiments, the body and projection are formed from mammalian heart valve tissue or pericardial tissue. In preferred embodiments, the body and projection are formed from porcine, bovine, or equine pericardial tissue.

Also provided are methods of reestablishing advantageous flow patterns such as sinus vortices in the aortic root in patients with ventricular assist device (VAD) support and a closed aortic valve orifice, by inserting the invention prosthesis into the aortic root so that aortic valve and the projection points downstream towards the aortic arch in the left ventricular outflow tract, and attaching the prosthesis to the closed aortic valve, thereby reestablishing reestablishing advantageous flow patterns such as sinus vortices in the aortic root.

Finally, there are provided methods of reducing thrombosis formation in patients with ventricular assist device (VAD) support and a closed aortic valve orifice by inserting the invention prosthesis into the aortic root so that aortic valve and the projection points downstream towards the aortic arch in the left ventricular outflow tract, and attaching the prosthesis to the closed aortic valve, thereby reducing thrombosis formation.

### BRIEF DESCRIPTION OF THE DRAWINGS

FIG. 1 shows a diagram of an exemplary embodiment of the valve prosthesis with flange feature for attachment to the heart.

FIG. 2 shows a diagram of an exemplary embodiment of the valve prosthesis with flange feature for attachment to the heart, and further including a gasket.

FIG. 3 shows a diagram of an exemplary embodiment of the valve prosthesis with flange feature for attachment to the heart, where the surface of the projection portion of the prosthesis is rough.

FIGS. 4A and 4B show the flow patterns in the mock circulatory loop with either a Flat valve (4A) or D1 valve (4B) at a proximal anastomosis, LVAD 9 krpm. The figures show that at the aortic root, the flat valve generates a circulating vortex, while the D1 valve generates small flow jet.

FIGS. 5A and 5B show the flow patterns in the mock circulatory loop with either a Flat valve (5A) and D1 valve (5B) at medial anastomosis, LVAD 9 krpm. The figures show that the flow patterns at the aortic root are significantly attenuated compared to the proximal anastomosis.

FIGS. 6A and 4B show the flow patterns in the mock circulatory loop with either a Flat valve (6A) and D1 valve (6B) at distal anastomosis, LVAD 9 krpm.

FIG. 7 shows a plot of the stagnation indexes against LVAD speed for each valve and anastomosis scenario. A high value for I_{S} indicates high fluid flow and low stagnation.

### DETAILED DESCRIPTION OF THE INVENTION

The present invention is based on the utilization of a prosthetic device in a surgical procedure in patients with ventricular assist device (VAD) support, in which the aortic valve orifice is sewn shut (i.e., closed off). The device consists of a prosthetic inserted during surgery into the aortic root that provides a flow-compatible geometry and surface designed to reestablish the formation of normal/advantageous fluid flow patterns. The formation of, for example, fluid vortices in the aortic root is considered important in the prevention of thrombus formation; thus, reestablishing advantageous flow patterns such as sinus vortices is one goal of the prosthetic design. The prosthesis will approximate the geometry of a native closed aortic valve, with a roughly conical shape that points downstream towards the aortic arch. Thus, the roughly conical shape of the prosthesis approximates the size and shape of an aortic valve in the closed position.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

The term “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

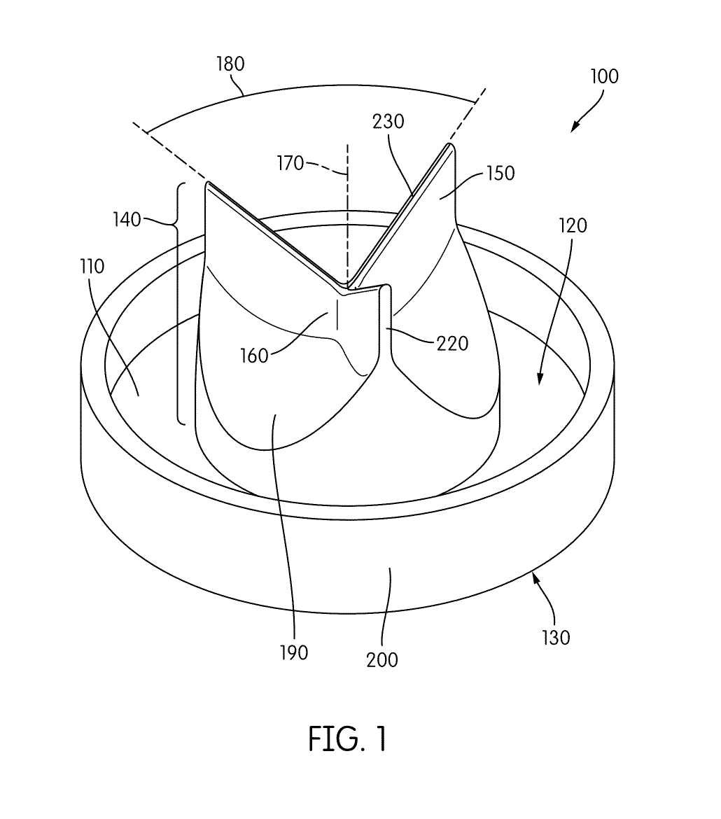

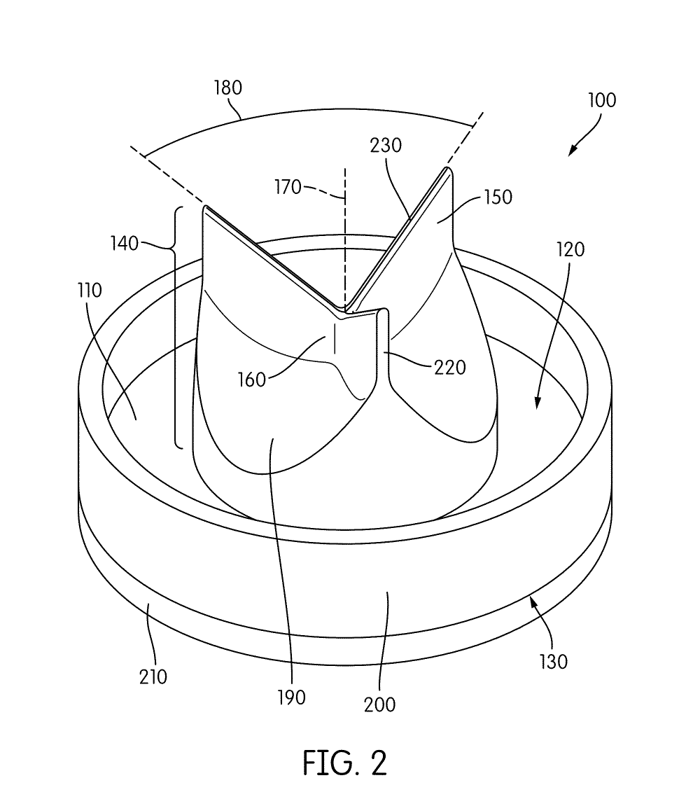

Referring to FIG. 1, an exemplary embodiment of the cardiac valve prosthesis 100 is shown. The prosthesis includes a circular body 110 and a projection 140. The circular body 110 has a first surface 120 and a second surface 130. The circular body 110 may further contain a flange 200 disposed along the circumferential edge of the body and extending away from the first surface of the body. The flange 200 is useful for attachment of the device to the native aortic valve. In some embodiments, the flange includes a sewing ring or gasket 210 (FIG. 2) that allows the prosthesis to be sutured directly to the tissue to fix it in place. While FIG. 2 shows the flange 200 and sewing ring or gasket 210 as having different thicknesses in the direction extending away from the first surface of the body, it should be understood that the flange 200 and sewing ring or gasket 210 may have the same thickness. In certain embodiments, the sewing ring is Dacron-covered.

The projection 140 is disposed on the first surface 120 of the circular body 110. The prosthesis 100 is designed to approximate the geometry of the native aortic valve when it is in the closed position. As such, in some embodiments the projection 140 has a roughly conical shape with a larger diameter at the end of the projection 140 that is in contact with the first surface 120 of the circular body 110, and a smaller diameter at the opposite or top end of the projection 140. In other embodiments, the projection 140 may be cylindrical in shape, having the same diameter at the end of the projection 140 that is in contact with the first surface 120 of the circular body 110 as the diameter at the opposite or top end of the projection 140.

In various embodiments, the projection 140 contains a plurality of walls 150 extending away from the first surface 120 of the circular body 110. Each wall 150 may be positioned such that a first side edge 160 contacts a center 170 of the body 110, thereby forming a plurality of intercepted arcs 180, each located between adjacent walls 150. Adjacent walls 150 are joined by a concave parabolic surface 190 that is disposed within each intercepted arc 180. In some embodiments, a second side edge 220 of each wall 150, which is the edge of the wall 150 on the outer circumference of the projection 140, inclines toward the center 170 of the projection 140. The incline is angled to provide the projection 140 with a generally conical shape.

Each wall 150 includes a top edge 230. In some embodiments the top edge 230 of the wall 150 inclines from the center 170 of the projection 140 toward the circumferential edge of the body 110. The top edge 230 may be flat or rounded. In various embodiments, the top edge 230 is rounded.

In some embodiments, the projection 140 has a finned, tri-leaflet geometry. In this embodiment, there are three walls 150 that come together at the center 170 of the projection 140 with an intercepted arc 180 of approximately 120 degrees.

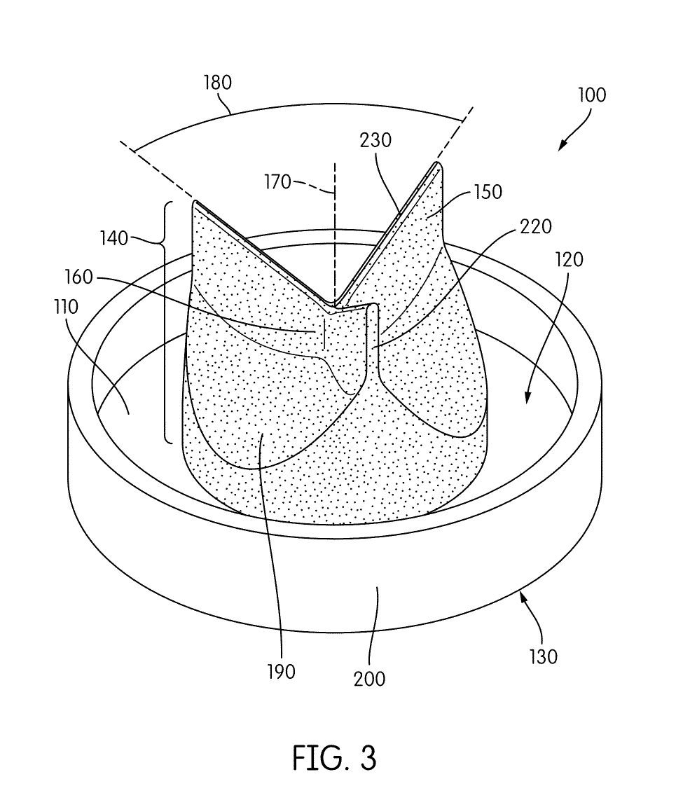

The projection 140 may have either a smooth surface or a rough surface. A smooth surface, as depicted in FIGS. 1 and 2, may be useful to discourage the nucleation of thrombi, while a rough surface, as depicted in FIG. 3, may be useful to encourage formation of a neointima or tissue covering of the surface of the projection 140, similar to what is seen in vascular grafts.

The device 100 may be formed from inert, biocompatible materials known to the skilled artisan for making implantable devices. For example, the device may be formed from a biological tissue, e.g., bovine pericardium, porcine pericardium, porcine valve, equine pericardium, bovine jugular vein, etc.; a synthetic or polymer material; a metallic material, e.g., a nickel-titanium alloy sheet made by a special process; or a tissue engineered valve. In some embodiments the synthetic or polymer material is polyurethane (PU), expanded polytetrafluroethylene (ePTFE) or pyrolytic carbon. The device may be made from a single material into a solid, yet pliable device. In various embodiments, the rigidity of the base may be higher than the finned projection 140 in order to support attachment to the tissue and not allow buckling. In other embodiments the device may be formed from biological tissue, such as pericardial tissue. In various embodiments the pericardial tissue is bovine, porcine, or equine.

In other embodiments, the device 100 can be formed for deployment via catheter (percutaneous implantation). For example, the device 100 is contained within a self-expanding frame (not shown) that supports the projection 140 and circular body 110. The projection 140 may be formed from a material such as porcine, bovine, equine or other mammalian pericardial tissue and may be affixed to the frame to provide support so that it maintains its shape upon implantation.

In various embodiments, the device 100 is formed from a plurality of materials. For example, glutaraldehyde-fixed bovine pericardial tissue may be used for the blood-contacting surface, Dacron polyester may be used for the flange 200 or sewing ring, and plastic or nitinol may be used for the frame or struts when present. These materials promote endothelialization of the various surfaces of the device 100. In other embodiments, a material such as pyrolytic carbon or other material that repels proteins may be used to form the projection 140 in order to keep the various surfaces free of cells or clots.

As discussed above, the device 100 may be used in conjunction with a closed mammalian cardiac valve, such as a human cardiac valve. Preferably the device 100 is used in conjunction with an aortic valve that has been sewn shut. In other embodiments, the device 100 may be used with a pulmonary valve.

The device 100 may be inserted during surgery, either during open heart surgery or inserted percutaneously, into the aortic root. It is inserted such that the device is positioned so that the second surface 130 of the circular body 110 faces the surgically closed aortic valve and the projection 140 points downstream towards the aortic arch in the left ventricular outflow tract. Thus, in various embodiments, the second surface 130 is in contact with the closed aortic valve. The device 100 may be affixed by suturing the flange 200 of the device 100 to the closed valve. In certain embodiments, the device 100 may be inserted percutaneously through the chest using a wand onto which the device 100 is mounted, and delivered through a lateral approach, thereby utilizing a less invasive surgical approach.

In embodiments in which the device 100 is inserted via a catheter, the device includes a self-expanding frame that may be compressed to a contracted delivery configuration onto an inner member of a delivery catheter. The device 100 and inner member may then be loaded into a delivery sheath of conventional design. The delivery catheter and device 100 are then advanced in a retrograde manner through a cut-down to the femoral artery and into the patient's descending aorta. The catheter then is advanced, under fluoroscopic guidance, over the aortic arch, through the ascending aorta to the surgically closed aortic valve. Once positioning of the catheter is confirmed, the sheath of the delivery catheter may be withdrawn proximally, thereby permitting the device 100 to self-expand. The device 100 may then be sutured into place.

The following examples are intended to illustrate but not limit the invention.

### EXAMPLE 1

This study was designed to test the hypothesis that aortic valve closure surgery, as currently practiced, produces abnormal aortic root flow patterns that lack the sinus vortices and instead exhibit regions of stagnation that reduce aortic washout, and that a prosthesis shaped like the closed aortic valve and designed to fit over the aortic valve orifice will reestablish advantageous flow patterns such as sinus vortex formation, and result in a consistent and improved outcome compared with the current techniques. The aims of the study were to (1) quantitatively measure the flow field in the aortic root of a LVAD-assisted heart in a cardiac simulator using Digital Image Particle Velocimetry (DPIV), and (2) use this method to compare the circulation with two different prosthetic valve closure designs.

A mock circulatory loop, which included a glass aorta connected to an LVAD (HEARTMATE II LVAD, Throatec Corporation), was designed to simulate flow in the aorta while supported by an LVAD, in order to investigate the fluid dynamics of the closed aortic valve. The glass aorta with physiologically representative dimensions allowed visualization of the flow field with a particle imaging velocimetry (PIV) system consisting of a CCD camera and a double-pulsed Nd:YAG laser (532 nm) that illuminated fluorescent tracer particles.

Two valve geometries were produced in silicone rubber. The first was a flat geometry (i.e., “flat valve” (simulates a surgically closed aortic valve)), typical of the current surgical practice; the second was a finned tri-leaflet geometry (i.e., “D1 valve” (novel prosthesis design)). The D1 valve prosthesis was 3D rapid prototyped (Fortus 400 mc), and injection molded using TC-5040 silicone rubber (BJB Enterprises). The two valves were placed in the aortic valve position with a circular gasket below to prevent any backflow toward the left ventricle. Three anastomosis positions on the model aorta were used: proximal, medial, and distal to the aortic valve. Several different flow conditions were investigated by connecting the LVAD outflow cannula to either proximal, medial, or distal positions along the aortic arch and by using a range of LVAD speeds from 8-12 krpm (i.e., 8, 9, 10, 11, and 12 krpm). A pulsatility pump simulated native heart function with settings of “On” simulating 72 bpm or “Off”, representing minimal cardiac function. The flow field was seeded with fluorescent particles and visualized using a LaVision Particle Image Velocimetry (PIV) system analyzed for the velocity field with DaVis software. Pressure and flow were recorded continuously at 200 Hz using LabChart (AD Instruments) during imaging.

Data were acquired, processed and analyzed using the DaVis (LaVision, Goettingen Germany) software, which captured and processed PIV images, and LabChart 7 Pro (AD Instruments) software, which recorded pressure and flow data. A sub-region of the flow field at the aortic root was quantified for stagnation (I_{S}) according to the following equation:

$${I}_{S} = \sqrt{\frac{{\int }_{{Ω}_{V}} {\vert u \vert}^{2} d Ω}{{\int }_{{Ω}_{V}} d Ω}}$$

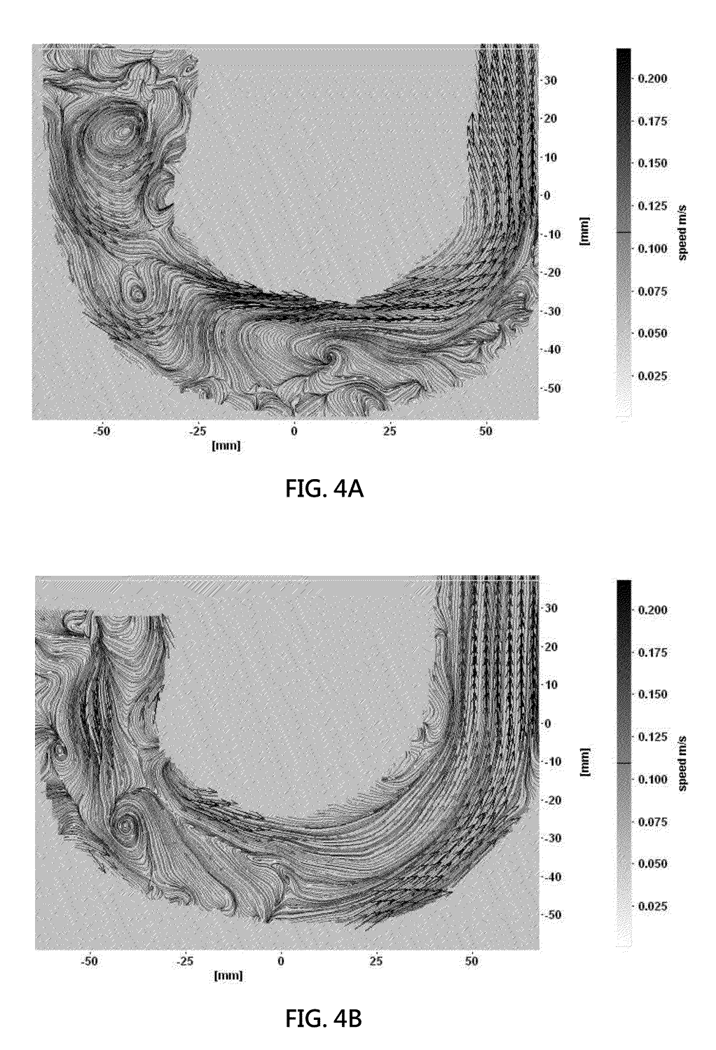

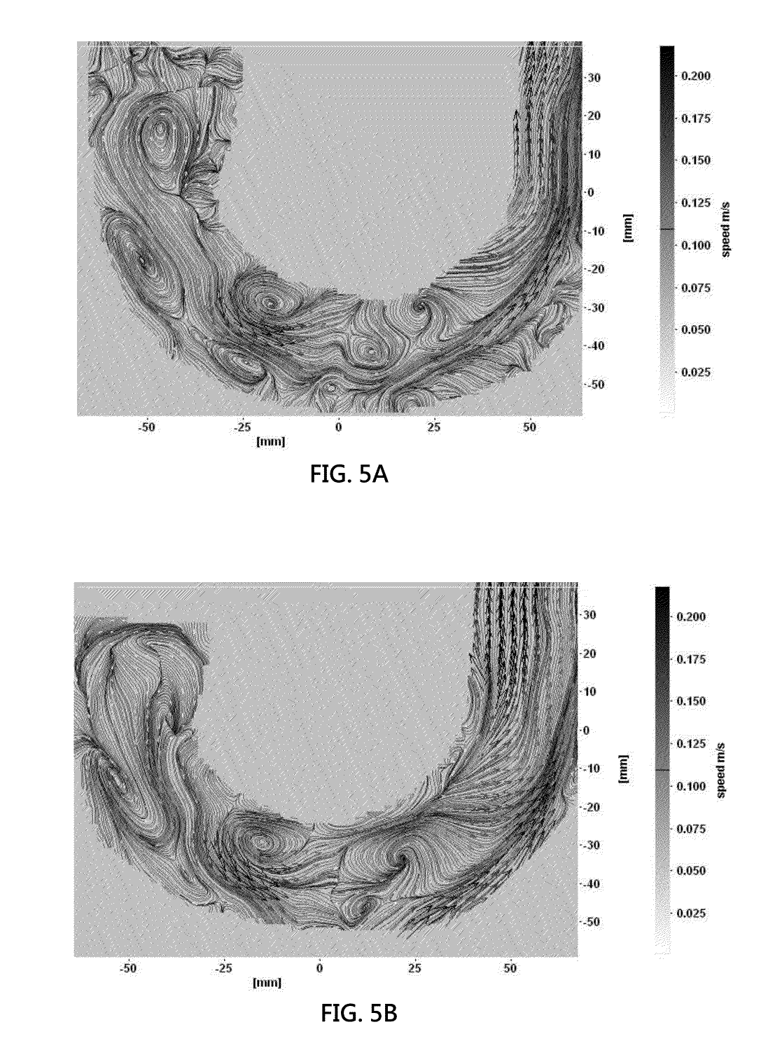

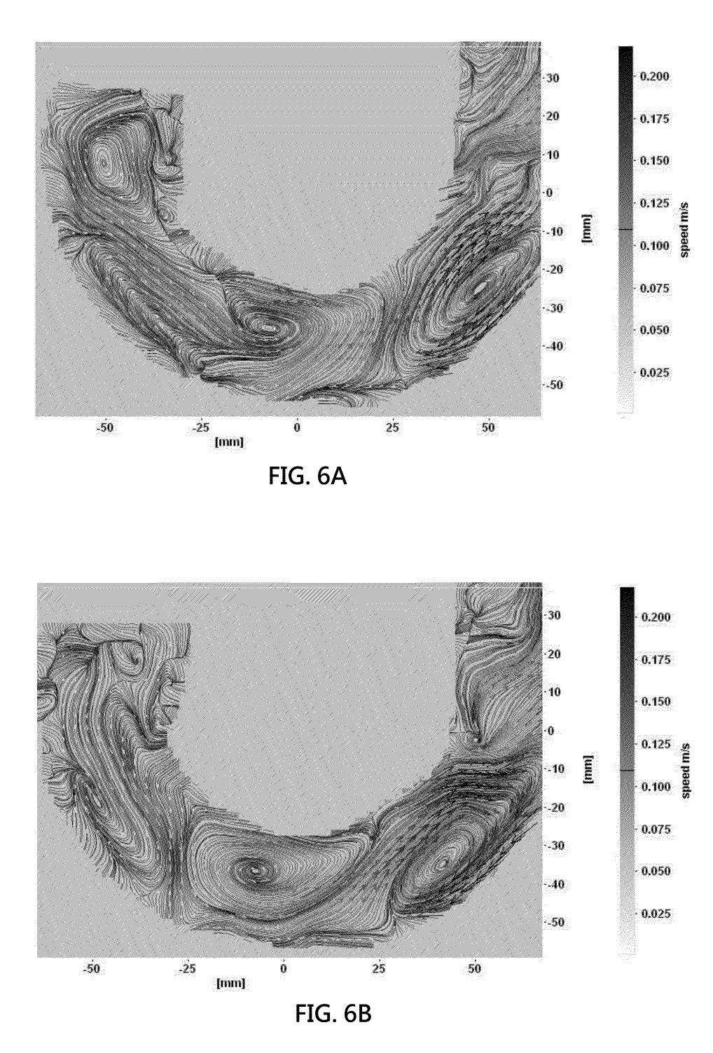

The cardiac output increased linearly through the range of VAD speeds, however the general flow pattern and profile appeared unchanged for 9-12 krpm. In the proximal anastomosis, the D1 valve generated a characteristic jet of fluid similar to normal antegrade flow (FIG. 4A). This effect was significantly diminished at the medial (FIG. 5A) and distal (FIG. 6A) anastomosis, likely due to the distanced inlet cannula. Additional PIV data for the above and below the mid-plane of the aorta (not shown) showed the D1 caused fluid to form small vortices in the aortic sinuses. These vortices are absent in the flat valve scenario. The I_{S} for D1 in the proximal position increased with LVAD speed likely due to an open flow pathway instead of a closed circular vortex. The difference in I_{S} was insignificant between the medial and distal positions.

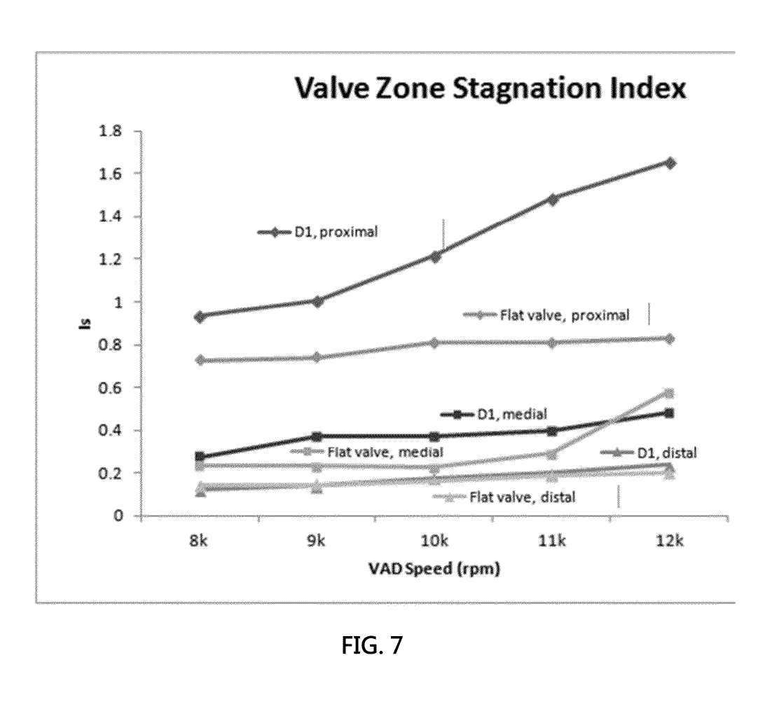

The Flat valve produced a low velocity, circular vortex centered at the aortic root (FIG. 4B). The D1 prosthesis eliminated the vortex and replaced it with a narrow, but relatively high velocity jet parallel to normal antegrade blood flow. Retrograde flow and local recirculation were observed along the length of the aorta when the cannula is connected to the medial (FIG. 5B) and distal (FIG. 6B) positions. A stagnation index was calculated for the aortic root, and the results shown in FIG. 7. These data indicate that the finned prosthesis improved the fluid dynamics of the aortic root with a proximal conduit connection, but the effects were significantly attenuated when the cannula is at the medial and distal positions. This mock circulatory loop study showed that aortic valve closure may be improved by using a finned prosthesis, which reduced flow stasis in the aortic root.

Although the invention has been described with reference to the above example, it will be understood that modifications and variations are encompassed within the spirit and scope of the invention. Accordingly, the invention is limited only by the following claims.

What is claimed is:

1. A cardiac prosthesis comprising:
(a) a circular body having a first surface and a second surface;
(b) a projection disposed on the first surface of the body, the projection comprising:
(i) a plurality of walls extending away from the first surface of the body, each wall being positioned such that a first side edge contacts a center of the body, thereby forming a plurality of intercepted arcs, each located between adjacent walls; and
(ii) a plurality of concave parabolic surfaces, each disposed within an intercepted arc,
wherein the projection is substantially shaped as a closed cardiac valve.

2. The cardiac prosthesis of claim 1, further comprising a flange disposed along a circumferential edge of the body and extending away from the first surface of the body.

3. The cardiac prosthesis of claim 1, further comprising a gasket disposed on the second surface of the body and configured to sealingly attach the body to a cardiac valve.

4. The cardiac prosthesis of claim 3, wherein the cardiac valve is a human cardiac valve.

5. The cardiac prosthesis of claim 1, wherein a second side edge of each wall inclines toward the center of the body.

6. The cardiac prosthesis of claim 5, wherein the projection is substantially conical.

7. The cardiac prosthesis of claim 1, wherein a top edge of each wall inclines from the center of the body toward the circumferential edge of the body.

8. The cardiac prosthesis of claim 1, wherein the projection comprises three walls.

9. The cardiac prosthesis of claim 1, wherein the top edge of each wall is rounded.

10. The cardiac prosthesis of claim 1, wherein the surface of the projection is smooth.

11. The cardiac prosthesis of claim 1, wherein the surface of the projection is rough.

12. The cardiac prosthesis of claim 1, wherein the body and projection are formed from a synthetic inert material.

13. The cardiac prosthesis of claim 1, wherein the body and projection are formed from mammalian pericardial tissue.

14. The cardiac prosthesis of claim 1, wherein the body and projection are formed from porcine, bovine, or equine pericardial tissue.

15. The cardiac prosthesis of claim 1, wherein the projection is formed to include a finned, tri-leaflet geometry.

16. A method of reestablishing normal fluid flow patterns in the aortic root of a subject having a surgically closed aortic valve comprising inserting the cardiac prosthesis of claim 1 into the aortic root of the subject and attaching the prosthesis to the closed aortic valve, thereby reestablishing reestablishing normal flow patterns in the aortic root.

17. The method of claim 16, wherein the prosthesis is positioned such that the projection points downstream towards an aortic arch in the left ventricular outflow tract of the subject.
